(19) United States
(12) Patent Application Publication
(10) Pub. No.: US 2026/0168691 A1
(43) Pub. Date: Jun. 18, 2026

(54) DEVICE, METHOD AND SYSTEM FOR CONTROLLING ENVIRONMENTAL CONTROL DEVICES

(71) Applicant: MOTOROLA SOLUTIONS, INC., Chicago, IL (US)

(72) Inventors: Jacob Shem TOV, Jerusalem (IL); Dragosh-Stefan SHARAGA, Airport City (IL); Grzegorz JANIK, Krakow (PL); Nir COHEN, Airport City (IL); Refael TZIDON, Airport City (IL); Nina BRONSTEIN, Airport City (IL); Pawel WILKOSZ, Myslenica (PL); Witold PLEWAKO, Krakow (PL); Sigalit LEYBMAN, Airport City (IL); Mateusz ZBYRADOWSKI, Jarosaw (PL); Michal KAIM, Mogilany (PL)

(21) Appl. No.: 18/985,946

(22) Filed: Dec. 18, 2024

(51) Int. Cl. F24F 11/63; F24F 11/49; F24F 120/10

(52) U.S. Cl. CPC F24F 11/63; F24F 11/49; F24F 2120/10

(57) ABSTRACT

A computing device is associated with an environment, and is communicatively coupled to: one or more environmental control devices located at the environment; and one or more sensors located at the environment. The computing device determines an environmental threshold associated with a medical condition of a user, and the one or more sensors are for sensing whether environmental conditions meet the environmental threshold. The computing device determines that the user is in the environment and, when the environmental conditions fail to meet the environmental threshold, the computing device controls the one or more environmental control devices to operate with the one or more sensors in a feedback loop, to change the environmental conditions until the environmental threshold is met.

### BACKGROUND OF THE INVENTION

Persons with certain medical conditions may be restricted from entering certain environments due to environmental conditions in the environment that may exacerbate their medical conditions. Indeed, when such an environment includes a hospital or a medical office, restricting such persons from entering the environments may lead to their medical conditions not getting treated or getting worse.

### BRIEF DESCRIPTION OF THE SEVERAL VIEWS OF THE DRAWINGS

In the accompanying figures similar or the same reference numerals may be repeated to indicate corresponding or analogous elements. These figures, together with the detailed description, below are incorporated in and form part of the specification and serve to further illustrate various embodiments of concepts that include the claimed invention, and to explain various principles and advantages of those embodiments.

FIG. 1 is a system for controlling environmental control devices, in accordance with some examples.

FIG. 2 is a device diagram showing a device structure of a device for controlling environmental control devices, in accordance with some examples.

FIG. 3 is a flowchart of a process for controlling environmental control devices, in accordance with some examples.

FIG. 4 depicts the system of FIG. 1 implementing a process for controlling environmental control devices, in accordance with some examples.

FIG. 5 depicts the system of FIG. 1 continuing to implement a process for controlling environmental control devices, in accordance with some examples.

FIG. 6 depicts the system of FIG. 1 continuing to implement a process for controlling environmental control devices, in accordance with some examples.

FIG. 7 depicts the system of FIG. 1 continuing to implement a process for controlling environmental control devices, in accordance with some examples.

Skilled artisans will appreciate that elements in the figures are illustrated for simplicity and clarity and have not necessarily been drawn to scale. For example, the dimensions of some of the elements in the figures may be exaggerated relative to other elements to help improve understanding of embodiments of the present disclosure.

The system, apparatus, and method components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

### DETAILED DESCRIPTION OF THE INVENTION

Certain persons may be sensitive to air quality, for example due to extreme cases of asthma. When such persons enter an environment, such as a hospital or mall where their doctor's office is located, and the air quality is poor, their asthma may cause such persons to experience trouble breathing, and the like, and may cause them to leave before getting treated. More generally, certain persons may have medical conditions that may restrict them from visiting environments where environmental conditions exacerbate and/or trigger their medical conditions.

Thus, there exists a need for an improved technical method, device, and system for controlling environmental control devices.

An aspect of the present specification provides a method comprising: determining, at a computing device, an environmental threshold associated with a medical condition of a user; determining, at the computing device, that the user is in an environment associated with the computing device, the computing device communicatively coupled to: one or more environmental control devices located at the environment; and one or more sensors located at the environment, the one or more sensors for sensing whether environmental conditions meet the environmental threshold; and when the environmental conditions fail to meet the environmental threshold, controlling, at the computing device, the one or more environmental control devices to operate with the one or more sensors in a feedback loop, to change the environmental conditions until the environmental threshold is met.

Another aspect of the present specification provides a computing device associated with an environment, the computing device comprising: a controller the communicatively coupled to: one or more environmental control devices located at the environment; and one or more sensors located at the environment; and a computer-readable storage medium having stored thereon program instructions that, when executed by the controller, causes the controller to perform a set of operations comprising: determining an environmental threshold associated with a medical condition of a user, the one or more sensors for sensing whether environmental conditions meet the environmental threshold; determining that the user is in the environment; and when the environmental conditions fail to meet the environmental threshold, controlling the one or more environmental control devices to operate with the one or more sensors in a feedback loop, to change the environmental conditions until the environmental threshold is met.

Each of the above-mentioned embodiments will be discussed in more detail below, starting with example system and device architectures of the system in which the embodiments may be practiced, followed by an illustration of processing blocks for achieving an improved technical method, device, and system for controlling environmental control devices.

Example embodiments are herein described with reference to flowchart illustrations and/or block diagrams of methods, apparatus (systems) and computer program products according to example embodiments. It will be understood that each block of the flowchart illustrations and/or block diagrams, and combinations of blocks in the flowchart illustrations and/or block diagrams, can be implemented by computer program instructions and/or program code and/or computer program code. These computer program instructions and/or program code may be provided to a processor of a general purpose computer, special purpose computer, or other programmable data processing apparatus to produce a special purpose and unique machine, such that the instructions, which execute via the processor of the computer or other programmable data processing apparatus, create means for implementing the functions/acts specified in the flowchart and/or block diagram block or blocks. The methods and processes set forth herein need not, in some embodiments, be performed in the exact sequence as shown and likewise various blocks may be performed in parallel rather than in sequence. Accordingly, the elements of methods and processes are referred to herein as “blocks” rather than “steps.”

These computer program instructions and/or program code may also be stored in a computer-readable memory that can direct a computer or other programmable data processing apparatus to function in a particular manner, such that the instructions stored in the computer-readable memory produce an article of manufacture including instructions which implement the function/act specified in the flowchart and/or block diagram block or blocks.

The computer program instructions and/or program code may also be loaded onto a computer or other programmable data processing apparatus that may be on or off-location, or may be accessed via the cloud in any of a software as a service (SaaS), platform as a service (PaaS), or infrastructure as a service (IaaS) architecture so as to cause a series of operational blocks to be performed on the computer or other programmable apparatus to produce a computer implemented process such that the instructions which execute on the computer or other programmable apparatus provide blocks for implementing the functions/acts specified in the flowchart and/or block diagram block or blocks. It is contemplated that any part of any aspect or embodiment discussed in this specification can be implemented or combined with any part of any other aspect or embodiment discussed in this specification.

Further advantages and features consistent with this disclosure will be set forth in the following detailed description, with reference to the drawings.

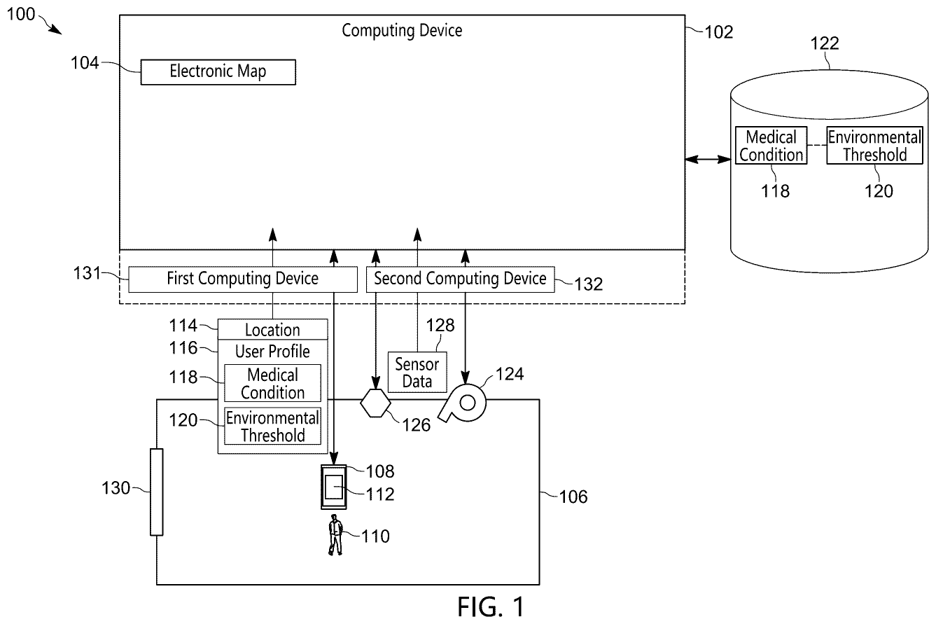

Attention is directed to FIG. 1, which depicts an example system 100 for controlling environmental control devices. The various components of the system 100 are in communication via any suitable combination of wired and/or wireless communication links, and communication links between components of the system 100 are depicted in FIG. 1, and throughout the present specification, as double-ended arrows between respective components; the communication links may include any suitable combination of wireless and/or wired links and/or wireless and/or wired communication networks.

The system 100 comprises a computing device 102, which, as depicted, stores an electronic map 104 of a given environment 106 (hereinafter interchangeably referred to as the environment 106 for simplicity).

The electronic map 104 may have any suitable format that may enable the computing device 102 to determine a location of a communication device 108, carried by a user 110, in the environment 106.

For example, as depicted, the communication device 108 is carried by a user 110, and includes an application 112 installed therein that may facilitate communication with the computing device 102. Indeed, as depicted, the communication device 108, via the application 112, is transmitting, to the computing device 102, a location 114, and the location 114 may be periodically provided to the computing device 102. For example, the communication device 108 may comprise a location determining device, such as Global Positioning System (GPS) device, which may be used by the communication device 108 to determine a respective location 114 (e.g., in GPS coordinates, and the like), and periodically report the location 114 to the computing device 102. In particular, the location 114 may be compared to the electronic map 104 to indicate a position of the communication device 108 within the environment 106.

As depicted, the communication device 108 may further transmit, to the computing device 102, a user profile 116 (e.g., hereafter, for simplicity, the profile 116) which includes an indication of a medical condition 118 of the user 110 and an indication of an environmental threshold 120 associated with the medical condition 118 of the user 110. While hereafter the present specification refers to the medical condition 118 of the user 110, it is understood that the medical condition 118 comprises an indication of the medical condition 118 of the user 110, such as a textual description, and the like. For example, the medical condition 118 may be indicated as “asthma”, “light sensitivity”, “noise sensitivity”, and the like, amongst other possibilities.

It is further understood that the environmental threshold 120, described in further detail below, may not be a component of the profile 116. Rather, in some examples, as depicted, the computing device 102 may be communicatively coupled to a memory 122 that may store the medical condition 118 in association with the environment threshold 120 such that the computing device 102 may receive the profile 116 indicating the medical condition 118, and retrieve the environment threshold 120 from the memory 122 using the medical condition 118 indicated in the profile 116.

While only one medical condition 118 and associated environment threshold 120 is depicted, it is understood that the memory 122 may store a plurality of medical conditions 118 and associated environment thresholds 120.

The environment 106 is further understood to include one or more environmental control devices 124 communicatively coupled to the computing device 102. The one or more environmental control devices 124 may be controlled by the computing device 102 to change environmental conditions at the environment 106.

The environment 106 is further understood to include one or more sensors 126 communicatively coupled to the computing device 102. The one or more sensors 126 sense environmental conditions at the environment 106 and provide, to the computing device 102, sensor data 128 indicative of the environmental conditions.

Hereafter, while reference may be made to only one environmental control device 124 and one sensor 126, it is understood that the system 100 may comprise any suitable number of environmental control devices 124 and sensors 126, for example distributed throughout the environment 106. Furthermore, one sensor 126 may include a plurality of sensors that sense different types of environmental conditions, such as air quality, lighting, and noise.

Furthermore, it is understood that environmental control devices 124 and sensors 126 may be provided in pairs, and that an environmental control device 124 may be controlled to change environmental conditions at the environment 106 in an area monitored by an associated sensor 126.

For example, when the environmental control device 124 is configured to control air quality in an area monitored by the sensor 126, such as a heating, ventilation, and air conditioning (HVAC) device, and/or air filtering device, that may filter pollutants and/or particulates, the sensor 126 may be configured to measure air quality in the area.

Similarly, when the environmental control device 124 is a noise-making device, such as the aforementioned HVAC device, and/or air filtering device, which may emit noise while heating, venting or cooling, and/or filtering, and which may be controlled to reduce (or increase) noise in an area monitored by the sensor 126, the sensor 126 may be configured to measure noise in the area.

Similarly, when the environmental control device 124 is configured to control lighting to increase or decrease in brightness in an area monitored by the sensor 126, the sensor 126 may be configured to measure brightness, and the like, in the area.

Indeed, as will be described herein, the computing device 102 may control the environmental control device 124 to change environmental conditions at the environment 106, in an area monitored by the sensor 126, until the environment threshold 120 is met, as indicated by the sensor data 128 from the sensor 126.

The environment 106 may comprise any suitable location that may be monitored, at least partially, using the computing device 102, including, but not limited to, a hotel, a mall a school, a restaurant, an arena, a convention center, a museum, an office building, amongst other possibilities. While FIG. 1 depicts only one room and/or space of the environment 106, it is understood that the environment 106 may comprise any suitable number of rooms and/or spaces, which may depend on an overall size of the environment 106.

It is understood that the environment 106 and the computing device 102 may be associated with an entity that manages the environment 106, and/or assists at managing the environment 106, such as a business, and the like.

In some examples, the computing device 102 may comprise one or more servers and/or one or more cloud computing devices that controls environmental conditions of the environment 106 as described herein.

The communication device 108 may comprise a mobile device and/or a cell phone and/or a tablet, and the like, that is carried by the user 110.

It is furthermore understood that the communication device 108 and/or application 112 may be registered with the computing device 102, and that the computing device 102 has been authorized to manage the environmental conditions at the environment 106 according to the environmental threshold 120.

Furthermore, during a registration process, the profile 116 may be provided to the computing device 102, which may store and/or cache the profile 116 at the memory 122. As such, when the user 110 arrives at the environment 106, the communication device 108, via the application 112, may notify the computing device 102 of the location 114, along with an identifier of the user profile 116.

When the location 114 indicates that the user 110 is in an area monitored by the sensor 126, whose environmental conditions are controllable via the environmental control device 124, the computing device 102 may control the environmental control device 124 to change environmental conditions at the environment 106, in an area of the sensor 126, until the environment threshold 120 is met, as indicated by the sensor data 128 from the sensor 126, in a feedback loop.

For example, when the medical condition of the user 110 is asthma, the environment threshold 120 may indicate a maximum concentration (e.g., in parts per million (PPM)) of pollutants and/or particulates that may be in the air to reduce the possibility of the user 110 having a medical incident due to a reaction to the pollutants and/or particulates. In these examples, and presuming the environmental control device 124 may be controlled to improve air quality (e.g., reduce the concentration of pollutants and/or particulates in the air by filtering the air), and further presuming that the sensor 126 senses concentration of pollutants and/or particulates, the environmental control device 124 may be controlled until the sensor data 128 indicates the concentration of pollutants and/or particulates in the air is at or below the maximum concentration of pollutants and/or particulates.

Similarly, when the medical condition of the user 110 is noise sensitivity, the environment threshold 120 may indicate a maximum noise level that reduces the possibility of the user 110 having a medical incident (e.g., mental distress) due to noise that is above the maximum noise level. In these examples, and presuming the environmental control device 124 may be controlled to reduce noise, and further presuming that the sensor 126 senses noise, the environmental control device 124 may be controlled until the sensor data 128 indicates that noise is below the maximum noise level.

Similarly, when the medical condition of the user 110 is light sensitivity, the environment threshold 120 may indicate a maximum brightness of light to reduce the possibility of the user 110 having a medical incident due to light that is above the maximum brightness. In these examples, and presuming the environmental control device 124 may be controlled to reduce brightness, and further presuming that the sensor 126 senses brightness, the environmental control device 124 may be controlled until the sensor data 128 indicates that brightness is below the maximum brightness.

Indeed, such control is understood to be in response to determining that the user 110 is in the environment 106, and such control is understood to end when the user 110 leaves the environment 106. Presence of the user 110 may be determined from the location 114, that may be periodically provided to the computing device 102, however absence of the user 110 may be determined by the computing device 102 no longer receiving the location 114.

Alternatively, or in addition, the user 110 may have operated the communication device 108 to provide an image of the user 110 to the computing device 102 (e.g., in the profile 116). The environment 106 may comprise cameras, communicatively coupled to the computing device 102, that may acquire and provide images to the computing device 102. The computing device 102 may compare such images to the image of the user 110, and determine presence or absence of the user 110 in the environment 106 via the image of the user 110 being present, or not present, in the images from the cameras. Indeed, a location of the user 110 in the environment 106 may be determined by the computing device 102 when the image of the user 110 is found in images from a camera located at a predetermined location. Alternatively, or in addition, the cameras may be configured to search for the image of the user 110 in images acquired by a given camera, and report to the computing device 102 when the image of the user 110 is found.

Alternatively, or in addition, the environment 106 may comprise an access point 130, such as a door, gate, and the like, that may include a card reader, and/or a ticket reader, and the like, communicatively coupled to the computing device 102, at which the user 110 may provide an access card, a ticket, and the like, to cause the access point 130 to unlock and/or open, and/or which may otherwise indicate that the user 110 is entering the environment 106. Hence, while not depicted, it is understood that there may be a communication link between the access point 130 and the computing device 102. In these examples, the access point 130 may communicate with the computing device 102 to indicate that the user 110 has entered the environment 106. In these examples, a location of the user 110 may be subsequently determined by the computing device 102 via the location 114 reported by the communication device 108, and/or the aforementioned cameras.

Indeed, in any of these examples, the computing device 102 may determine not only presence or absence of the user 110 in the environment 106, but a location of the user 110 in the environment 106. Indeed, the location of the user 110 in the environment 106, and in particular relative to locations of the environmental control devices 124 and the sensors 126, may be performed using the electronic map 104, which may also have indicated therein locations of the environmental control devices 124 and the sensors 126 (and any cameras, when present).

It is further understood that, as depicted, the computing device 102 may comprise one or more of: a first computing device 131 and a second computing device 132. The computing devices 102, 131, 132 may be combined in one enclosure, or may be physically separated from one another, with functionality described herein distributed therebetween. As such, as depicted, the computing device 102 extends using broken lines to encompass the computing devices 131, 132, the broken lines indicating that the computing devices 102, 131, 132 may be combined in one enclosure, or may be physically separated from one another.

The first computing device 131 may be associated with the application 112 for communicating with the communication device 108 of the user 110, and hence communications between the communication device 108 and the computing device 102 may be via the first computing device 131. As such, depicted, a communication link between the communication device 108 and the computing device 102 may be via the first computing device 131. Indeed, first computing device 131 may comprise one or more servers and/or one or more cloud computing devices that manages services associated with the application 112.

The second computing device 132 may be associated with the environment 106. For example, the second computing device 132 may comprise one or more servers local to the environment 106 that controls the one or more environmental control devices 124 for example in conjunction with the one or more sensors 126 and any devices associated with the environment 106 which may be used to set environmental conditions.

For example, when an environmental control device 124 comprises an HVAC device that may control temperature, a sensor 126 may comprise a temperature sensor and the environment 106 may further comprise a thermostat (e.g., which may be incorporated into the temperature sensor), at which a temperature for the environment 106 may be selected by an administrator of the system 100 and/or the environment 106. The second computing device 132, in the absence of controlling the HVAC device according to the environmental threshold 120, may control the HVAC device according to a setting at the thermostat. More generally, the second computing device 132, in a normal mode, may control an environmental control device 124 according to locally set environmental targets (e.g., such as a target temperature). However, controlling an environmental control device 124 according to the environmental threshold 120 is understood to override control in the normal mode, and the like. As such, as depicted, respective communication links between the environmental control device 124 and the sensor 126, and the computing device 102 may be via the second computing device 132. Indeed, while not depicted, a respective communication link between the access point 130 and the computing device 102 may be via the second computing device 132, as the second computing device 132 may be further configured to unlock and/or open the access point 130 on the basis of an access card and/or ticket presented by the user 110.

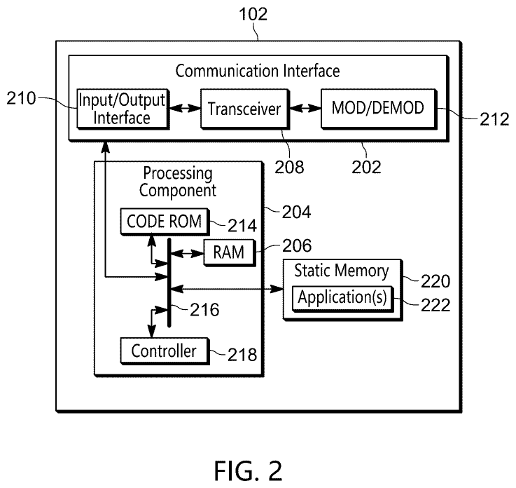

Attention is next directed to FIG. 2, which depicts a schematic block diagram of an example of the computing device 102.

While the computing device 102 is depicted in FIG. 2 as a single component, the computing device 102 may be distributed among a plurality of components (e.g., such as the computing devices 131, 132) and the like including, but not limited to, any suitable combination of one or more servers, one or more cloud computing devices, and the like. In some examples, the computing device 102 and a sensor 126 may be combined and/or partially combined, and/or the computing device 102 and an environmental control device 124 may be combined and/or partially combined.

As depicted, the computing device 102 comprises: a communication interface 202, a processing component 204, a Random-Access Memory (RAM) 206, one or more wireless transceivers 208 (which may be optional), one or more wired and/or wireless input/output (I/O) interfaces 210, a combined modulator/demodulator 212, a code Read Only Memory (ROM) 214, a common data and address bus 216, a controller 218, and a static memory 220 storing at least one application 222. Hereafter, the at least one application 222 will be interchangeably referred to as the application 222.

In some examples, the memory 220 may be combined with the memory 122 and/or the medical conditions 118 and the associated environmental thresholds 120 may be stored at the memory 220.

While not depicted, the computing device 102 may include one or more of an input device and a display screen and the like.

As shown in FIG. 2, the computing device 102 includes the communication interface 202 communicatively coupled to the common data and address bus 216 of the processing component 204.

The processing component 204 may include the code Read Only Memory (ROM) 214 coupled to the common data and address bus 216 for storing data for initializing system components. The processing component 204 may further include the controller 218 coupled, by the common data and address bus 216, to the Random-Access Memory 206 and the static memory 220.

The communication interface 202 may include one or more wired and/or wireless input/output (I/O) interfaces 210 that are configurable to communicate with other components of the system 100, such as the communication devices 108, the environmental control devices 124, the sensors 126, and the access point 130.

For example, the communication interface 202 may include one or more transceivers 208 and/or wireless transceivers for communicating with other suitable components of the system 100. Hence, the one or more transceivers 208 may be adapted for communication with one or more communication links and/or communication networks used to communicate with the other components of the system 100. For example, the one or more transceivers 208 may be adapted for communication with one or more of the Internet, a digital mobile radio (DMR) network, a Project 25 (P25 ) network, a terrestrial trunked radio (TETRA) network, a Bluetooth network, a Wi-Fi network, for example operating in accordance with an IEEE 802.11 standard (e.g., 802.11a, 802.11b, 802.11g), an LTE (Long-Term Evolution) network and/or other types of GSM (Global System for Mobile communications) and/or 3GPP (3^{rd} Generation Partnership Project) networks, a 5G network (e.g., a network architecture compliant with, for example, the 3GPP TS 23 specification series and/or a new radio (NR) air interface compliant with the 3GPP TS 38 specification series) standard), a Worldwide Interoperability for Microwave Access (WiMAX) network, for example operating in accordance with an IEEE 802.16 standard, and/or another similar type of wireless network.

Hence, the one or more transceivers 208 may include, but are not limited to, a cell phone transceiver, a DMR transceiver, P25 transceiver, a TETRA transceiver, a 3GPP transceiver, an LTE transceiver, a GSM transceiver, a 5G transceiver, a Bluetooth transceiver, a Wi-Fi transceiver, a WiMAX transceiver, and/or another similar type of wireless transceiver configurable to communicate via a wireless radio network.

However, at least a digital mobile radio (DMR) network, a Project 25 (P25 ) network, a terrestrial trunked radio (TETRA) network and any corresponding DMR transceiver, P25 transceiver, and TETRA transceiver may be dedicated for use by first responder radios and/or any other first responder communication devices. In these examples, the communication device 108 may comprise a first responder radios and/or communication device, and the user 110 may be a first responder, such as a police officer, a fire fighter, an emergency medical technician, and the like.

The communication interface 202 may further include one or more wireline transceivers 208, such as an Ethernet transceiver, a USB (Universal Serial Bus) transceiver, or similar transceiver configurable to communicate via a twisted pair wire, a coaxial cable, a fiber-optic link, or a similar physical connection to a wireline network. The transceiver 208 may also be coupled to a combined modulator/demodulator 212.

The controller 218 may include ports (e.g., hardware ports) for coupling to other suitable hardware components of the system 100.

The controller 218 may include one or more logic circuits, one or more processors, one or more microprocessors, one or more GPUs (Graphics Processing Units), and one or more TPUs (Tensor Processing Units), and the like, and/or the controller 218 may include one or more ASIC (application-specific integrated circuits) and one or more FPGA (field-programmable gate arrays), and/or another electronic device. In some examples, the controller 218 and/or the computing device 102 is not a generic controller and/or a generic device, but a device specifically configured to implement functionality for controlling environmental control devices. For example, in some examples, the computing device 102 and/or the controller 218 specifically comprises a computer executable engine configured to implement functionality for controlling environmental control devices.

The static memory 220 comprises a non-transitory machine readable medium that stores machine readable instructions to implement one or more programs or applications and/or program code. Example machine readable media include a non-volatile storage unit (e.g., Erasable Electronic Programmable Read Only Memory (“EEPROM”), Flash Memory) and/or a volatile storage unit (e.g., random-access memory (“RAM”)). In the example of FIG. 2, programming instructions (e.g., machine readable instructions) that implement the functionality of the computing device 102 as described herein are maintained, persistently, at the memory 220 and used by the controller 218, which makes appropriate utilization of volatile storage during the execution of such programming instructions.

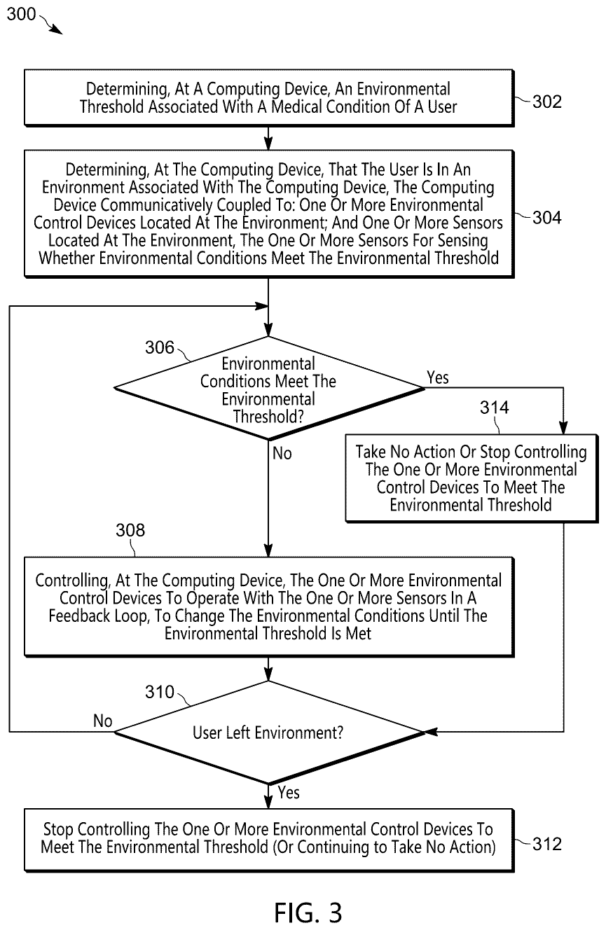

In particular, the memory 220 stores instructions and/or program code corresponding to the at least one application 222 that, when executed by the controller 218, enables the controller 218 to implement functionality for controlling environmental control devices, including but not limited to, the blocks of the process set forth in FIG. 3.

The application 222 may include programmatic algorithms, and the like, to implement functionality as described herein.

Alternatively, and/or in addition to programmatic algorithms, the application 222 may include one or more machine learning algorithms to implement functionality as described herein.

The one or more machine learning algorithms of the application 222 may include, but are not limited to: a deep-learning based algorithm; a neural network; a generalized linear regression algorithm; a random forest algorithm; a support vector machine algorithm; a gradient boosting regression algorithm; a decision tree algorithm; a generalized additive model; evolutionary programming algorithms; Bayesian inference algorithms, reinforcement learning algorithms, and the like. Any suitable machine learning algorithm and/or deep learning algorithm and/or neural network is within the scope of present examples.

When one or more machine learning algorithm are used to implement functionality as described herein, the one or more machine learning algorithm may be trained to control an environmental control device 124 to meet an environmental threshold 120 using positive training data and/or negative training data.

For example, positive training data for controlling an environmental control device 124 to meet an environmental threshold 120 may include positive training input comprising sensor data that indicates that the environmental threshold 120 is not met, as well as the environmental threshold 120, and positive training output may comprise one or more commands to control the environmental control device 124 to change environmental conditions at the environment 106 (e.g., in an area of the sensor 126) to meet the environmental threshold 120, and/or that improve the environmental conditions relative to the environmental threshold 120. Such commands may be manually determined, or may be determined using a programmatic algorithm, and the like.

Similarly, negative training data for controlling an environmental control device 124 to meet an environmental threshold 120 may include negative training input comprising sensor data that indicates that the environmental threshold 120 is not met, as well as the environmental threshold 120, and negative training output may comprise one or more commands to control the environmental control device 124 to change environmental conditions at the environment 106 (e.g., in an area of the sensor 126) that continue to cause the environmental threshold 120 not to be met, and/or that make the environmental conditions worse relative to the environmental threshold 120. Such commands may be manually determined, or may be determined using a programmatic algorithm, and the like.

While details of other components of the system 100 are not depicted, such as the communication device 108, the communication device 108 may have a structure similar to that of the computing device 102, but adapted for respective functionality of the communication device 108. For example, the communication devices 108 are understood to comprise a display screen, a speaker, a microphone, and one or more input devices, in addition to the components depicted in FIG. 2, for example for interaction with the application 112.

Attention is now directed to FIG. 3, which depicts a flowchart representative of a process 300 for controlling environmental control devices. The operations of the process 300 of FIG. 3 correspond to machine readable instructions that are executed by the computing device 102, and specifically the controller 218 of the computing device 102. In the illustrated example, the instructions represented by the blocks of FIG. 3 are stored at the memory 220 for example, as the application 222. The process 300 of FIG. 3 is one way in which the controller 218 and/or the computing device 102 and/or the system 100 may be configured. Furthermore, the following discussion of the process 300 of FIG. 3 will lead to a further understanding of the system 100, and its various components.

The process 300 of FIG. 3 need not be performed in the exact sequence as shown and likewise various blocks may be performed in parallel rather than in sequence. Accordingly, the elements of process 300 are referred to herein as “blocks” rather than “steps.” The process 300 of FIG. 3 may be implemented on variations of the system 100 of FIG. 1, as well.

At a block 302, the controller 218, and/or the computing device 102, determines an environmental threshold 120 associated with a medical condition 118 of a user 110.

For example, at the block 302, determining the environmental threshold 120 may comprise the controller 218, and/or the computing device 102, one or more of: receiving, from a communication device 108 associated with the user 110, the environmental threshold 120; and receiving, from the communication device 108, an indication of the medical condition 118; and determining the environmental threshold 120 from the indication.

The environmental threshold 120 may be a component of a user profile 116 associated with the user 110 and/or may be stored at the memory 122. The profile 116 may be received in a registration process and/or the profile 116 may be received with the location 114, for example when the user 110 enters the environment 106 with the communication device 108.

Furthermore, while the medical condition 118 has been described with respect to asthma, light sensitivity and noise sensitivity, the medical condition 118 may comprise any suitable type of medical condition that may be exacerbated by, and/or sensitive to, certain environmental conditions that may be controllable at the environment 106. Furthermore, the examples of asthma, light sensitivity and noise sensitivity, indicate that the medical condition 118 (e.g., an indication of the medical condition 118) may not be provided in a format that specifically indicates the sensitivity to an environmental condition, such as “light sensitivity” and “noise sensitivity” but rather the medical condition 118 may be indicated by a medical term, such as “asthma”, that has an associated sensitivity to an environmental condition (e.g., sensitive to pollutants and/or particulates, and the like).

Furthermore, the medical condition 118 may include a physical medical condition, a mental health condition, a psychological health condition, and the like.

At a block 304, the controller 218, and/or the computing device 102, determines that the user 110 is in the environment 106 associated with the computing device 102, the computing device 102 communicatively coupled to: one or more environmental control devices 124 located at the environment 106; and one or more sensors 126 located at the environment 106, the one or more sensors 126 for sensing whether environmental conditions meet the environmental threshold 120.

In some examples, as has already been described, determining, at the block 304, that the user 110 is in the environment 106 may comprise the controller 218, and/or the computing device 102, one or more of: receiving, from the communication device 108 associated with the user 110, a location 114 of the user 110; and comparing the location 114 with an electronic map 104 associated with the environment 106; receiving, from the communication device 108 associated with the user 110, an indication that the user 110 is in the environment 106; and receiving, from an access point 130 associated with the environment 106, the indication that the user 110 is in the environment 106.

At a block 306, the controller 218, and/or the computing device 102, determines whether the environmental conditions fail to meet the environmental threshold 120.

For example, the sensor data 128, that represents an environmental condition, may be compared with a corresponding environmental threshold 120, to determine whether or not the environmental threshold 120 has been met.

For example, in the case of air quality, when the sensor data 128 indicates that the concentration of pollutants and/or particulates are greater than a maximum concentration indicated by an environmental threshold 120, the controller 218, and/or the computing device 102, may determine that the environmental conditions fail to meet the environmental threshold 120. Conversely, when the sensor data 128 indicates that the concentration of pollutants and/or particulates are less than a maximum concentration indicated by an environmental threshold 120, the controller 218, and/or the computing device 102, may determine that the environmental conditions meet the environmental threshold 120.

In another example, when the sensor data 128 indicates that brightness of light is greater than a maximum brightness indicated by an environmental threshold 120, the controller 218, and/or the computing device 102, may determine that the environmental conditions fail to meet the environmental threshold 120. Conversely, when the sensor data 128 indicates that brightness of light is less than a maximum brightness indicated by an environmental threshold 120, the controller 218, and/or the computing device 102, may determine that the environmental conditions meet the environmental threshold 120.

In another example, when the sensor data 128 indicates that a noise level is greater than a maximum noise level indicated by an environmental threshold 120, the controller 218, and/or the computing device 102, may determine that the environmental conditions fail to meet the environmental threshold 120. Conversely, when the sensor data 128 indicates that a noise level is less than a maximum noise level indicated by an environmental threshold 120, the controller 218, and/or the computing device 102, may determine that the environmental conditions meet the environmental threshold 120.

Other examples are within the scope of the present specification. For example, a sensor 126 may be configured to sense a particular gas, such as carbon dioxide, and/or any other suitable particular gas, which may exacerbate a medical condition such as asthma, and an environmental control device 124 may comprise an HVAC device, and/or air filtering device, that may filter carbon dioxide and/or any other suitable particular gas. Such particular gases may include, but are not limited to, the aforementioned carbon dioxide, volatile organic compounds, carbon monoxide, ozone, and the like. In these examples, when the sensor data 128 indicates that a particular gas level and/or concentration is greater than a maximum particular gas level and/or concentration indicated by an environmental threshold 120, the controller 218, and/or the computing device 102, may determine that the environmental conditions fail to meet the environmental threshold 120. Conversely, when the sensor data 128 indicates that a particular gas level and/or concentration is less than a maximum particular gas level and/or concentration indicated by an environmental threshold 120, the controller 218, and/or the computing device 102, may determine that the environmental conditions meet the environmental threshold 120.

Other examples are within the scope of the present specification. For example, a sensor 126 may be configured to sense temperature, which may also exacerbate certain medical conditions, and an environmental control device 124 may comprise an HVAC device that may reduce temperature. In these examples, when the sensor data 128 indicates that a temperature is greater than a maximum temperature indicated by an environmental threshold 120, the controller 218, and/or the computing device 102, may determine that the environmental conditions fail to meet the environmental threshold 120. Conversely, when the sensor data 128 indicates that a temperature level is less than a maximum temperature indicated by an environmental threshold 120, the controller 218, and/or the computing device 102, may determine that the environmental conditions meet the environmental threshold 120.

While such examples show that an environmental threshold 120 may not be met when the sensor data 128 indicates that an associated environmental condition is below an environmental threshold 120, in other examples, an environmental threshold 120 may not be met when an associated environmental condition is above an environmental threshold 120. For example, a medical condition 118 may indicate that it is preferable that the user 110 breathe air with oxygen levels above a minimum oxygen level indicated by an associated environmental threshold 120. In these examples, when the sensor data 128 indicates that an oxygen level and/or concentration is greater than a minimum oxygen level and/or concentration indicated by an environmental threshold 120, the controller 218, and/or the computing device 102, may determine that the environmental conditions meets the environmental threshold 120. Conversely, when the sensor data 128 indicates that an oxygen level and/or concentration is greater than a minimum oxygen level and/or concentration indicated by an environmental threshold 120, the controller 218, and/or the computing device 102, may determine that the environmental conditions fails to meet the environmental threshold 120. In such examples, the sensor 126 may sense such oxygen levels and/or concentrations, and the environmental control device 124 may be configured to pump oxygen into the environment 106 to increase an oxygen level and/or concentration.

It is further understood, however, that the environmental threshold 120 may be limited to be within certain parameters. Using temperature as an example, a maximum temperature received in the environmental threshold 120 of the user profile 116 may be less than a minimum temperature associated with the environment 106. To ensure that the environment 106 is not controlled to sub-zero temperatures, or to temperatures that are not compatible with HVAC devices of the environment 106, when a maximum temperature received in the environmental threshold 120 is less than a minimum temperature associated with the environment 106, the maximum temperature received in the environmental threshold 120 may be adjusted to the minimum temperature associated with the environment 106. Furthermore, the communication device 108 may be notified that is not possible to meet the maximum temperature received in the environmental threshold 120 so that the user 110 may decide whether to leave the environment 106 or stay in the environment 106. Similar examples apply with respect to air quality, light, sound, particular gases, and the like. For example, with light, it is understood that a minimum brightness may be maintained in the environment 106, even when a maximum brightness of an environmental threshold 120 is below the minimum brightness.

When the environmental conditions fail to meet the environmental threshold 120 (e.g., a “NO” decision at the block 306), at a block 308, the controller 218, and/or the computing device 102, controls the one or more environmental control devices 124 to operate with the one or more sensors 126 in a feedback loop, to change the environmental conditions until the environmental threshold 120 is met.

For example, the controller 218, and/or the computing device 102, may provide one or more respective commands to the one or more environmental control devices 124 to change to change the environmental conditions, for example to filter air (e.g., to remove pollutants and/or particulates and/or a particular gas, such as carbon dioxide), reduce brightness, reduce noise, reduce temperature, increase oxygen, amongst other possibilities.

At a block 310, the controller 218, and/or the computing device 102, determines whether the user 110 has left the environment 106.

For example, determining whether the user 110 has left the environment 106 may be similar to determining whether the user 110 is present in the environment 106, as has already been described.

When the user 110 has left the environment 106 (e.g., a “YES” decision at the block 310), at a block 312, the controller 218, and/or the computing device 102, stops controlling the one or more environmental control devices 124 to meet the environmental threshold 120.

Returning to the block 306, when the environmental conditions meet the environmental threshold 120 (e.g., a “YES” decision at the block 306), at a block 314, the controller 218, and/or the computing device 102, takes no action, but again determines whether the user 110 has left the environment 106 at the block 310.

Alternatively, when the block 308 has been previously implemented (e.g., the block 306 is again being implemented due to a “NO” decision at the block 310), and when the environmental conditions meet the environmental threshold 120 (e.g., a “YES” decision at the block 306), at an alternative implementation of the block 314, similar to the block 312, the controller 218, and/or the computing device 102, stops controlling the one or more environmental control devices 124 to meet the environmental threshold 120.

Returning to the block 310, when the user 110 has left the environment 106 (e.g., a “YES” decision at the block 310), and the controller 218, and/or the computing device 102, has previously stopped controlling the one or more environmental control devices 124 to meet the environmental threshold 120, in an alternative implementation of the block 312, the controller 218, and/or the computing device 102, continues to take no action.

Put another way, at the block 312, the controller 218, and/or the computing device 102 stops controlling the one or more one or more environmental control devices 124 to meet the environmental threshold 120 or continues to take no action, depending on whether or not the environmental conditions meet the environmental threshold 120 as determined at the block 306.

Returning to the block 310, when the user 110 has not left the environment 106 (e.g., a “NO” decision at the block 306), the controller 218, and/or the computing device 102, again determines whether the environmental conditions fail to meet the environmental threshold 120, and may again implement the block 308 when the environmental conditions fail to meet the environmental threshold 120, or implement the block 314 when the environmental conditions meet the environmental threshold 120.

Indeed, the repeating of the blocks 306, 308 generally represent control of the one or more environmental control devices 124 to operate with the one or more sensors 126 in a feedback loop.

The process 300 may include any other suitable features.

For example, the process 300 may further comprise, when the environmental conditions fail to meet the environmental threshold 120 (e.g., a “NO” decision at the block 306), the controller 218, and/or the computing device 102: providing, to the communication device 108 associated with the user 110, a notification of a failure to meet the environmental threshold 120; receiving, from the communication device 108, a command to proceed with controlling the one or more environmental control devices 124; and in response to receiving the command, performing the controlling the one or more environmental control devices 124 to operate with the one or more sensors 126 in the feedback loop, to change the environmental conditions until the environmental threshold 120 is met.

Put another way, when an environmental decision does not meet an environmental threshold 120, for example a “NO” decision occurs at the block 306, prior to implementing the block 308 to control the one or more environmental control devices 124, the controller 218, and/or the computing device 102 may notify the communication device 108 associated with the environmental threshold 120 and/or the medical condition 118 as to the failure to meet the environmental threshold 120.

The notification may include an electronic button that, when actuated at the communication device 108 by the user 110, causes the communication device 108 to transmit, to the computing device 102, a command to proceed with controlling the one or more environmental control devices 124. The notification may further include another electronic button that, when actuated at the communication device 108 by the user 110, causes the communication device 108 to transmit, to the computing device 102, a command to not proceed with controlling the one or more environmental control devices 124 and/or to cancel further implementation of the process 300.

Using an example of the medical condition 118 indicating that the user 110 is noise sensitive, the sensor 126 measuring noise, and an environmental control device 124 being controllable to reduce noise, the notification may indicate that noise levels are above a maximum noise level. The user 110 may actuate the electronic button that causes the communication device 108 to transmit, to the computing device 102, a command to continue with controlling of the environmental control device 124 to reduce noise. Alternatively, the user 110 may be having a day where their noise sensitivity is not acute, and the user 110 may actuate the electronic button that causes the communication device 108 to transmit, to the computing device 102, a command to cancel controlling of the environmental control device 124 to reduce noise and/or to take no action. Hence, depending on the command received at the computing device 102, the controller 218, and/or the computing device 102, may either proceed with implementing the block 308, or proceed to the block 312, regardless of the decision at the block 306.

Alternatively or in addition, the process 300 may further comprise, the controller 218, and/or the computing device 102, one of: storing the environmental threshold 120 associated with the medical condition 118 of the user 110 until the user 110 leaves the environment 106; and deleting the environmental threshold 120 associated with the medical condition 118 of the user 110 once the user 110 leaves the environment 106; or caching the environmental threshold 120 associated with the medical condition 118 of the user 110, after the user 110 leaves the environment 106, for later usage when the user 110 is again in the environment 106.

Put another way, the profile 116 and/or the medical condition 118 and/or the environmental threshold 120, and/or any personally identifiable information (PII) associated with the user 110, may be stored at the memory 122 while the user 110 is in the environment 106 and either deleted when the user 110 leaves the environment 106, or cached at the memory 122 for later usage when the user 110 is again in the environment 106. The deletion of the profile 116 and/or the medical condition 118 and/or the environmental threshold 120, and/or any PII associated with the user 110 may be performed to maintain privacy of the user 110. However the user 110 may alternatively preauthorize caching of such information for later usage (e.g., in a registration process), to reduce later bandwidth usage and processing of the profile 116 and/or the medical condition 118 and/or the environmental threshold 120, and/or any personally PII associated with the user 110 (e.g., when the profile 116 and/or the medical condition 118 and/or the environmental threshold 120, and/or any personally PII associated with the user 110 is later again provided) when the user 110 is again in the environment 106.

At the process 300 it is further understood that the computing device 102 may comprise one or more of: the first computing device 131 associated with an application 112 for communicating with a communication device 108 of the user 110; the second computing device 132 associated with the environment 106; and one or more cloud computing devices.

At the process 300 it is further understood that the one or more sensors 126 may be configured to detect one or more of: air quality; one or more particular gases; sound; light; amongst other possibilities.

At the process 300 it is further understood that the one or more environmental control devices 124 may comprise one or more of: an air flow control device; an HVAC device; an air filtering device; a noise-making device; a light emitting device; amongst other possibilities.

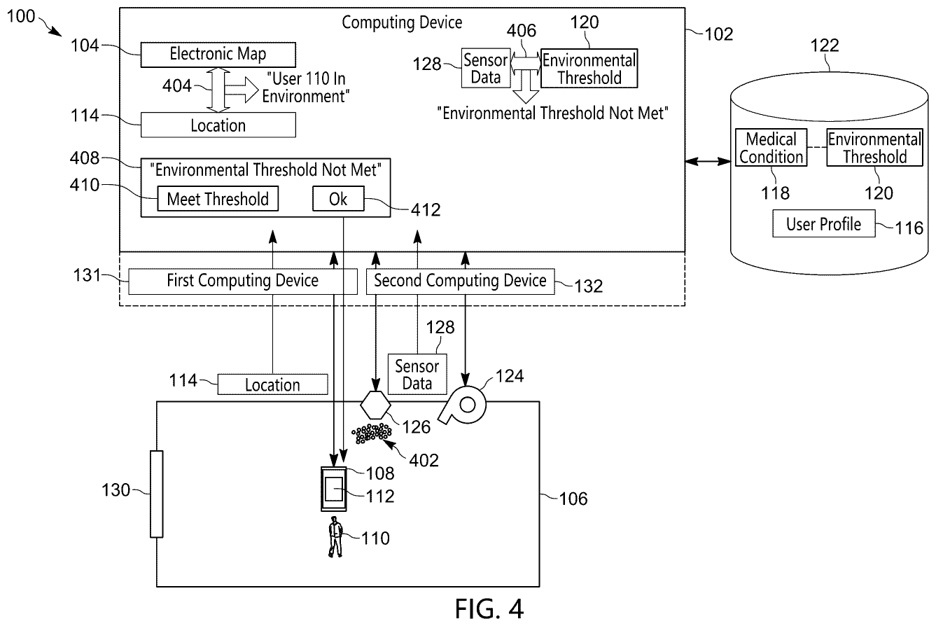

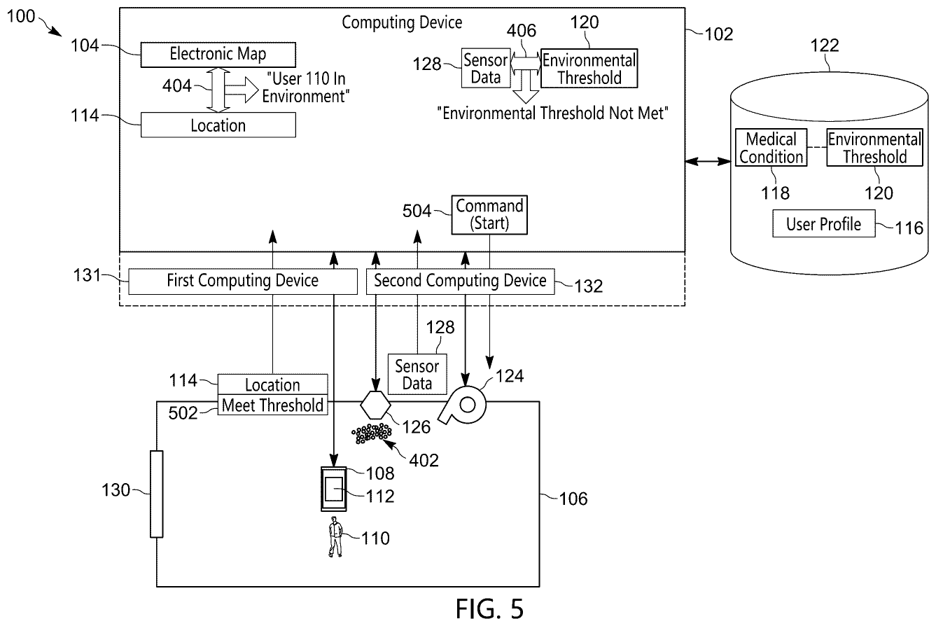

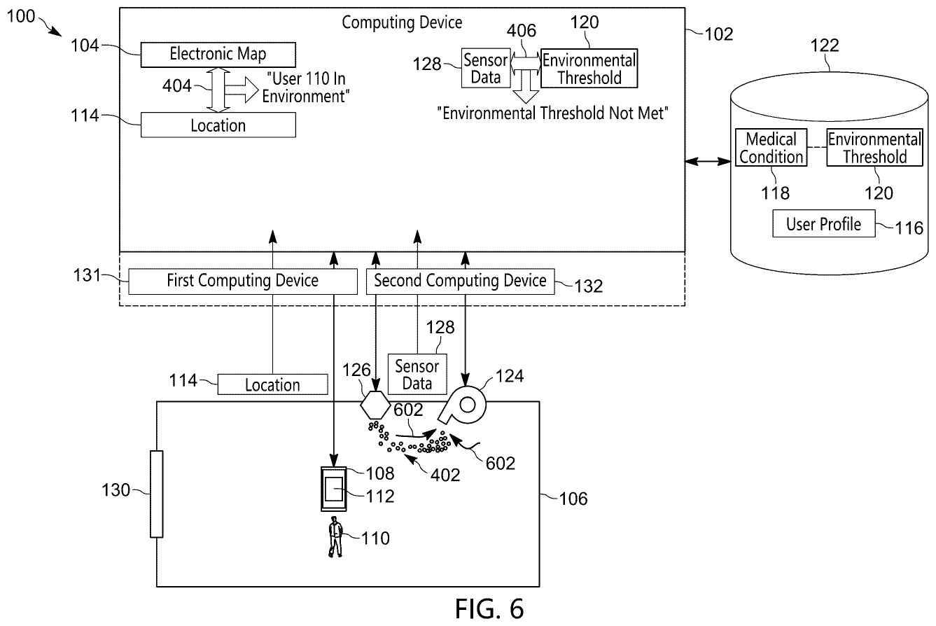

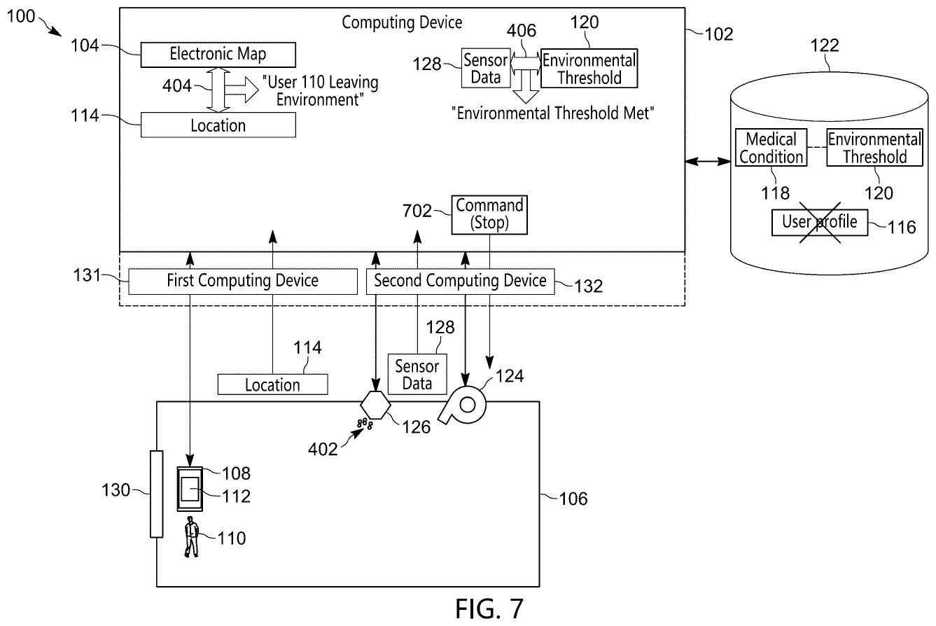

Attention is next directed to FIG. 4, FIG. 5, FIG. 6, and FIG. 7, which depict an example of the process 300 being implemented in the system 100. FIG. 4, FIG. 5, FIG. 6, and FIG. 7 are similar to FIG. 1, with like components having like numbers.

Furthermore, the description of FIG. 4, FIG. 5, FIG. 6, and FIG. 7 uses air quality as an example, and it is understood that the sensor 126 may detect concentration of pollutants and/or particulates in the air, and the environmental control device 124 may be controlled to remove, and/or filter, such pollutants and/or particulates from the air.

For example, in FIG. 4, such particulates 402 are depicted adjacent the sensor 126 and/or in an area monitored by the sensor 126, and the sensor data 128 is understood to indicate concentration, and the like, of the particulates 402.

FIG. 4 further depicts the user profile 116 cached at the memory 122, such that the computing device 102 has determined (e.g., at the block 302), the environmental threshold 120 associated with the medical condition 118 of the user 110.

FIG. 4 further depicts the computing device 102 comparing the location 114 to the electronic map 104 (e.g., as represented by an arrow 404 therebetween), and has determined (e.g., at the block 304 of the process 300) that the user 110 is in the environment 106, as represented by text “User 110 in Environment”.

FIG. 4 further depicts the computing device 102 comparing (e.g., at the block 306 of the process 300) the sensor data 128 with the environmental threshold 120 associated with the user 110 (e.g., as represented by an arrow 406 therebetween), which is understood to comprise and/or indicate a maximum concentration of the particulates 402. In particular, the computing device 102 has determined (e.g., a “NO” decision at the block 306 of the process 300) that the environmental threshold 120 has not been met, as represented by text “Environmental Threshold Not Met”. For example, the concentration of the particulates 402 as indicated in the sensor data 128 may be greater than the maximum concentration indicated by the environmental threshold 120.

In response to determining that the environmental threshold 120 has not been met, the computing device 102 generates a notification 408 of a failure to meet the environmental threshold (e.g., via text “Environmental Threshold Not Met”), and transmits the notification 408 to the communication device 108. The notification 408 may be provided at a display screen of the communication device 108, for example within the application 112. As depicted, the notification 408 includes a first electronic button 410 (e.g., also labelled “Meet Threshold”) that, when actuated at the communication device 108, causes the communication device 108 to transmit, to the computing device 102, a command to meet the environmental threshold 120. As depicted, the notification 408 includes a second electronic button 412 (e.g., also labelled “OK”) that, when actuated at the communication device 108, causes the communication device 108 to transmit, to the computing device 102, a command to take no action (e.g., and not meet the environmental threshold 120).

Attention is next directed to FIG. 5, which follows from FIG. 4, and depicts the communication device 108 transmitting, to the computing device 102, a command 502 (e.g., also labelled “Meet Threshold”) to meet the environmental threshold 120. Such an example assumes that the first electronic button 410 was actuated at the communication device 108. FIG. 5 further depicts the communication device 108 continuing to provide the location 114 (e.g., as the communication device 108 is carried, by the user 110, around the environment 106).

In response to receiving the command 502, the computing device 102 generates a command 504 that instructs the environmental control device 124 to “Start” operating to change the environmental conditions, for example to meet the environmental threshold 120. As depicted, the computing device 102 transmits the command 504 to the environmental control device 124 to control (e.g., at the block 308 of the process 300) the environmental control device 124 to operate to change the environmental condition until the environmental threshold 120 is met, for example to reduce the concentration of the particulate in an area monitored by the sensor 126, as described with respect to FIG. 6.

FIG. 5 further shows, however, that the sensor 126 continues to provide the sensor data 128 to the computing device 102, which continues to compare the sensor data 128 with the environmental threshold 120. Indeed, as depicted, the computing device 102 continues to determine that the environmental condition represented by the sensor data 128 indicates that the environmental threshold 120 is not met. It is understood, however, that if the computing device 102 determines that the environmental condition represented by the sensor data 128 indicates that the environmental threshold 120 is met, the command 504 may not be generated and/or transmitted.

Similarly, FIG. 5 further shows that the location 114 continues to be provided to the computing device 102 which continues to compare the location 114 with the electronic map 104. Indeed, as depicted,, the computing device 102 continues to determine that the user 110 is in the environment 106. It is understood, however, that if the computing device 102 determines that the user 110 has left the environment 106, the command 504 may not be generated and/or transmitted.

Attention is next directed to FIG. 6, which follows from FIG. 5, and which depicts the environmental control device 124 operating according to the command 502, for example to filter the particulates 402. In particular, the command 502 may control the environmental control device 124 to exit a current “normal” mode (e.g., in which air flow intake occurs and air is filtered at a predetermined air flow intake rate), and enter a filtering mode to increase air flow intake, as represented by arrows 602, to cause the particulates 402 to flow away from the sensor 126 and into the environmental control device 124, thereby reducing concentration of the particulates in an areas monitored by the sensor 126. The filtering mode may be different from the normal mode in that, in the filtering mode, air flow intake rate may be increased relative to the normal mode.

The sensor 126 continues to measure the concentration of the particulates 402 and provide corresponding sensor data 128 to the computing device 102, and the communication device 108 continues to provide the location 114 to the computing device 102.

Attention is next directed to FIG. 7, which follows from FIG. 6, and which depicts the concentration of the particulates 402 being reduced in an area monitored by the sensor 126, such that the sensor data 128 now indicates that the environmental condition represented by the sensor data 128 meets the environmental threshold 120, as represented by the text “Environmental Threshold Met”.

As such, the computing device 102 generates a command 702 that instructs the environmental control device 124 to “Stop” operating to change the environmental conditions, for example as the environmental threshold 120 has been met. As depicted, the computing device 102 transmits the command 702 to the environmental control device 124 to control (e.g., at the block 314 of the process 300) the environmental control device 124 to stop operating to change the environmental condition. For example, the environmental control device 124 is controlled to “Stop” filtering the particulates 402, and/or to return to the normal mode the environmental control device 124 was in prior to receiving the command 504.

FIG. 7 further depicts the user 110 carrying the communication device 108 towards the access point 130 to leave the environment 106, and the location 114 indicates such to the computing device 102. For example, the computing device 102 may continue to compare the location 114 to the electronic map 104 and determine that the user 110 is leaving the environment 106 (e.g., as indicated via text “User 110 Leaving Environment”). In this example, once the user 110 has left the environment 106 the computing device 102 may transmit the command 702 to the environmental control device 124 to control (e.g., at the block 312 of the process 300) the environmental control device 124 to stop operating to change the environmental condition, regardless of whether the environmental condition indicated by the sensor data 128 meets or does not meet the environmental threshold 120.

Indeed, the command 702 may be transmitted under either condition (e.g., the environmental threshold 120 has been met and/or the user 110 has left the environment 106 or is leaving the environment 106).

As further depicted in FIG. 7, the user profile 116 may be deleted from the memory 122, as represented by an “X” therethrough, when the user 110 has left the environment 106 or is leaving the environment 106, for example by way of the computing device 102 providing a delete command (not depicted) to the memory 122.

In yet alternative examples, the command 702 may be transmitted when the location 114 indicates that the user 110 has left an area monitored by the sensor 126, for example to enter another part of the environment 106. Indeed, presuming the environment 106 includes pairs of sensors 126 and environmental control devices 124 distributed throughout the environment 106, the computing device 102 may implement the process 300 with respect to given pairs of sensors 126 and environmental control devices 124 as the user 110 enters and exits areas monitored by respective sensors 126 of the given pairs of sensors 126 and environmental control devices 124. Indeed, changes in the location 114 may, in some examples, be used to predict where the user 110 may be heading, and the process 300 may be implemented with respect to given pairs of sensors 126 and environmental control devices 124 prior to the user 110 arriving at areas monitored by sensors 126 of the given pairs, to begin to control the environmental conditions to meet an environmental threshold 120 prior to the user 110 arriving at an area monitored by a sensor 126.

Furthermore, when more than one user 110 is in the environment 106, and respective associated environmental thresholds 120 are different (e.g., different maximum particulate concentrations), the computing device 102 may control an environmental control device 124 to meet the most extreme of the respective environmental thresholds 120 (e.g., a lowest maximum particulate concentration of the different maximum particulate concentrations) that minimizes exacerbation of both respective medical conditions 118.

Furthermore, when more than one user 110 is in the environment 106, and associated environmental thresholds 120 result in control of an environmental control device 124 to take actions that contradict each other (e.g., increase filtering by an air filtering device to reduce particulate concentration, and decrease filtering by an air filtering device to reduce noise emissions), the computing device 102 may use a weighting scheme to determine which action to take, which may depend on associated medical conditions 118 of the users 110. For example, the computing device 102 may determine that a medical condition 118 of “asthma” has a higher weight than a medical condition 118 of “noise sensitivity”. While the latter may result in uncomfortableness in the associated user 110, such uncomfortableness may not be life threatening, whereas a high concentration of particulates may be life threatening to a user 110 with asthma. In these examples, a communication device 108 of a user 110 whose associated environmental threshold 120 may not be met may be notified so that the user 110 may decide whether or not to stay in the environment 106. For example, the user 110 with noise sensitivity may be notified, via their communication device 108, that a user 110 with asthma is in the environment 106 and that noise may actually increase to accommodate the user 110 with asthma.

The processes and devices described herein may be adapted for other uses. For example, in some examples a crowd density threshold may be associated with a medical condition of a user; for example, a user may get panic attacks in dense crowds. A sensor of the environment 106 may comprise a crowd sensor, and/or any other suitable sensor that may measure numbers of people and/or crowd density, and the like, including, but not limited to, one or more cameras acquiring images to which video analytics, and the like, may be applied to determine a crowd density. When a crowd density of the environment 106 is above a maximum crowd density associated with a medical condition of the user, the computing device 102 may notify an associated communication device of the user. Alternatively, the computing device 102 may control speakers, and the like, at the environment 106 to attempt to get a crowd to disperse to accommodate the user. For example, the speaker may play a notice of “Patrons in Area A: we'd ask that some of you move to another area, at least temporarily. This is not an emergency, but merely an attempt to accommodate some other patrons with crowd sensitivity issues”. Hence, the computing device 102 may implement a feedback loop to continue to control crowd density, for example, by playing notices until the crowd density decreases below a maximum crowd density.

As should be apparent from this detailed description above, the operations and functions of the electronic computing device are sufficiently complex as to require their implementation on a computer system, and cannot be performed, as a practical matter, in the human mind. Electronic computing devices such as set forth herein are understood as requiring and providing speed and accuracy and complexity management that are not obtainable by human mental steps, in addition to the inherently digital nature of such operations (e.g., a human mind cannot interface directly with RAM or other digital storage, cannot control an environmental control device in a feedback loop with a sensor).

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. Unless the context of their usage unambiguously indicates otherwise, the articles “a,” “an,” and “the” should not be interpreted as meaning “one” or “only one.” Rather these articles should be interpreted as meaning “at least one” or “one or more.” Likewise, when the terms “the” or “said” are used to refer to a noun previously introduced by the indefinite article “a” or “an,” “the” and “said” mean “at least one” or “one or more” unless the usage unambiguously indicates otherwise.

Also, it should be understood that the illustrated components, unless explicitly described to the contrary, may be combined or divided into separate software, firmware, and/or hardware. For example, instead of being located within and performed by a single electronic processor, logic and processing described herein may be distributed among multiple electronic processors. Similarly, one or more memory modules and communication channels or networks may be used even if embodiments described or illustrated herein have a single such device or element. Also, regardless of how they are combined or divided, hardware and software components may be located on the same computing device or may be distributed among multiple different devices. Accordingly, in this description and in the claims, if an apparatus, method, or system is claimed, for example, as including a controller, control unit, electronic processor, computing device, logic element, module, memory module, communication channel or network, or other element configured in a certain manner, for example, to perform multiple functions, the claim or claim element should be interpreted as meaning one or more of such elements where any one of the one or more elements is configured as claimed, for example, to make any one or more of the recited multiple functions, such that the one or more elements, as a set, perform the multiple functions collectively.

It will be appreciated that some embodiments may be comprised of one or more generic or specialized processors (or “processing devices”) such as microprocessors, digital signal processors, customized processors and field programmable gate arrays (FPGAs) and unique stored program instructions and/or program code (including both software and firmware) that control the one or more processors to implement, in conjunction with certain non-processor circuits, some, most, or all of the functions of the method and/or apparatus described herein. Alternatively, some or all functions could be implemented by a state machine that has no stored program instructions and/or program code, or in one or more application specific integrated circuits (ASICs), in which each function or some combinations of certain of the functions are implemented as custom logic. Of course, a combination of the two approaches could be used.

Moreover, an embodiment can be implemented as a computer-readable storage medium having computer readable code stored thereon for programming a computer (e.g., comprising a processor) to perform a method as described and claimed herein. Any suitable computer-usable or computer readable medium may be utilized. Examples of such computer-readable storage mediums include, but are not limited to, a hard disk, a CD-ROM, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. In the context of this document, a computer-usable or computer-readable medium may be any medium that can contain, store, communicate, propagate, or transport the program for use by or in connection with the instruction execution system, apparatus, or device.

Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation. For example, computer program code for carrying out operations of various example embodiments may be written in an object oriented programming language such as Java, Smalltalk, C++, Python, or the like. However, the computer program code for carrying out operations of various example embodiments may also be written in conventional procedural programming languages, such as the “C” programming language or similar programming languages. The program code may execute entirely on a computer, partly on the computer, as a stand-alone software package, partly on the computer and partly on a remote computer or server or entirely on the remote computer or server. In the latter scenario, the remote computer or server may be connected to the computer through a local area network (LAN) or a wide area network (WAN), or the connection may be made to an external computer (for example, through the Internet using an Internet Service Provider).

The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “one of”, without a more limiting modifier such as “only one of”, and when applied herein to two or more subsequently defined options such as “one of A and B” should be construed to mean an existence of any one of the options in the list alone (e.g., A alone or B alone) or any combination of two or more of the options in the list (e.g., A and B together).

A device or structure that is “configured” in a certain way is configured in at least that way, but may also be configured in ways that are not listed.

The terms “coupled”, “coupling” or “connected” as used herein can have several different meanings depending on the context in which these terms are used. For example, the terms coupled, coupling, or connected can have a mechanical or electrical connotation. For example, as used herein, the terms coupled, coupling, or connected can indicate that two elements or devices are directly connected to one another or connected to one another through intermediate elements or devices via an electrical element, electrical signal or a mechanical element depending on the particular context.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

What is claimed is:

1. A method comprising:
determining, at a computing device, an environmental threshold associated with a medical condition of a user;
determining, at the computing device, that the user is in an environment associated with the computing device, the computing device communicatively coupled to: one or more environmental control devices located at the environment; and one or more sensors located at the environment, the one or more sensors for sensing whether environmental conditions meet the environmental threshold; and
when the environmental conditions fail to meet the environmental threshold, controlling, at the computing device, the one or more environmental control devices to operate with the one or more sensors in a feedback loop, to change the environmental conditions until the environmental threshold is met.

2. The method of claim 1, wherein determining the environmental threshold comprises one or more of:
receiving, from a communication device associated with the user, the environmental threshold; and
receiving, from the communication device, an indication of the medical condition; and determining the environmental threshold from the indication.

3. The method of claim 1, wherein the environmental threshold is a component of a user profile associated with the user.

4. The method of claim 1, wherein determining that the user is in the environment comprises one or more of:
receiving, from a communication device associated with the user, a location of the user; and comparing the location with an electronic map of the environment;
receiving, from the communication device associated with the user, an indication that the user is in the environment; and
receiving, from an access point associated with the environment, the indication that the user is in the environment.

5. The method of claim 1, further comprising, when the environmental conditions fail to meet the environmental threshold:
providing, to a communication device associated with the user, a notification of a failure to meet the environmental threshold;
receiving, from the communication device, a command to proceed with controlling the one or more environmental control devices; and
in response to receiving the command, performing the controlling the one or more environmental control devices to operate with the one or more sensors in the feedback loop, to change the environmental conditions until the environmental threshold is met.

6. The method of claim 1, further comprising one of:
storing the environmental threshold associated with the medical condition of the user until the user leaves the environment; and deleting the environmental threshold associated with the medical condition of the user once the user leaves the environment; or
caching the environmental threshold associated with the medical condition of the user, after the user leaves the environment, for later usage when the user is again in the environment.

7. The method of claim 1, further comprising:
determining that the user has left the environment; and, in response,
stop controlling the one or more environmental control devices to meet the environmental threshold.

8. The method of claim 1, wherein the computing device comprises one or more of:
a first computing device associated with an application for communicating with a communication device of the user;
a second computing device associated with the environment; and
one or more cloud computing devices.

9. The method of claim 1, wherein the one or more sensors are configured to detect one or more of:
air quality;
one or more particular gases;
sound; and
light.

10. The method of claim 1, the one or more environmental control devices comprise one or more of:
an air flow control device;
an HVAC (Heating, Ventilation, and Air Conditioning) device;
an air filtering device;
a noise-making device; and
a light emitting device.

11. A computing device associated with an environment, the computing device comprising:
a controller the communicatively coupled to: one or more environmental control devices located at the environment; and one or more sensors located at the environment; and
a computer-readable storage medium having stored thereon program instructions that, when executed by the controller, causes the controller to perform a set of operations comprising:
determining an environmental threshold associated with a medical condition of a user, the one or more sensors for sensing whether environmental conditions meet the environmental threshold;
determining that the user is in the environment; and
when the environmental conditions fail to meet the environmental threshold, controlling the one or more environmental control devices to operate with the one or more sensors in a feedback loop, to change the environmental conditions until the environmental threshold is met.

12. The computing device of claim 11, wherein determining the environmental threshold comprises one or more of:
receiving, from a communication device associated with the user, the environmental threshold; and
receiving, from the communication device, an indication of the medical condition; and determining the environmental threshold from the indication.

13. The computing device of claim 11, wherein the environmental threshold is a component of a user profile associated with the user.

14. The computing device of claim 11, wherein determining that the user is in the environment comprises one or more of:
receiving, from a communication device associated with the user, a location of the user; and comparing the location with an electronic map of the environment;
receiving, from the communication device associated with the user, an indication that the user is in the environment; and
receiving, from an access point associated with the environment, the indication that the user is in the environment.

15. The computing device of claim 11, wherein the set of operations further comprise, when the environmental conditions fail to meet the environmental threshold:
providing, to a communication device associated with the user, a notification of a failure to meet the environmental threshold;
receiving, from the communication device, a command to proceed with controlling the one or more environmental control devices; and
in response to receiving the command, performing the controlling the one or more environmental control devices to operate with the one or more sensors in the feedback loop, to change the environmental conditions until the environmental threshold is met.

16. The computing device of claim 11, wherein the set of operations further comprise one of:
storing the environmental threshold associated with the medical condition of the user until the user leaves the environment; and deleting the environmental threshold associated with the medical condition of the user once the user leaves the environment; or
caching the environmental threshold associated with the medical condition of the user, after the user leaves the environment, for later usage when the user is again in the environment.

17. The computing device of claim 11, wherein the set of operations further comprise:
determining that the user has left the environment; and, in response,
stop controlling the one or more environmental control devices to meet the environmental threshold.

18. The computing device of claim 11, wherein the computing device comprises one or more of:
a first computing device associated with an application for communicating with a communication device of the user;
a second computing device associated with the environment; and
one or more cloud computing devices.

19. The computing device of claim 11, wherein the one or more sensors are configured to detect one or more of:
air quality;
one or more particular gases;
sound; and
light.

20. The computing device of claim 11, the one or more environmental control devices comprise one or more of:
an air flow control device;
an HVAC (Heating, Ventilation, and Air Conditioning) device;
an air filtering device;
a noise-making device; and
a light emitting device.
